(19) United States
(12) Patent Application Publication
(10) Pub. No.: US 2010/0297112 A1
(43) Pub. Date: Nov. 25, 2010

(54) COMBINATIONS COMPRISING DMXAA FOR THE TREATMENT OF CANCER

(75) Inventors: Colin Green, London (GB); Lloyd Kelland, London (GB); Gail Rowlinson-Bruza, London (GB)

Correspondence Address: LAHIVE & COCKFIELD, LLP;FLOOR 30, SUITE 3000, ONE POST OFFICE SQUARE, BOSTON, MA, 02109, US

(73) Assignee: Antisoma Research Limited

(21) Appl. No.: 12/064,633

(22) PCT Filed: Aug. 25, 2006
(86) PCT No.: PCT/GB2006/003196

§ 371 Date: Aug. 5, 2009

(30) Foreign Application Priority Data
Aug. 26, 2005 (GB) 0517386.9
Mar. 2, 2006 (GB) 0604114.9

(51) Int. Cl. A61K 39/395; A61P 35/00; A61K 31/352

(52) U.S. Cl. 424/133.1; 424/141.1; 514/455

(57) ABSTRACT

The present invention relates to combinations of compounds such as compounds of the xanthenone acetic acid class such as 5,6-dimethylxanthenone-4-acetic acid (DMXAA) and vascular endothelial growth factor binders, in particular the monoclonal antibody Avastin™ (bevacizumab). More particularly, the invention is concerned with the use of such combinations in the treatment of cancer and pharmaceutical formulations containing such combinations.

The present invention relates to combinations of compounds of the class having the formula (I) as defined below, for example compounds of the xanthenone acetic acid class having the formula (II) as defined below, such as 5,6-dimethylxanthenone-4-acetic acid (DMXAA), or a pharmaceutically acceptable salt, ester or prodrug thereof and vascular endothelial growth factor (VEGF) binders, in particular the monoclonal antibody Avastin™ (bevacizumab). The combinations of compounds described above may also include a taxane, in particular paclitaxel or docetaxel. For example, the present invention relates to synergistic combinations of compounds of the class having the formula (I) as defined below, for example compounds of the xanthenone acetic acid class having the formula (II) as defined below, such as 5,6-dimethylxanthenone-4-acetic acid (DMXAA), or a pharmaceutically acceptable salt, ester or prodrug thereof and anti-angiogenic growth factor inhibitors, in particular the monoclonal antibody Avastin™ (bevacizumab), a VEGF binder and such combinations may also include a taxane, in particular paclitaxel or docetaxel. More particularly, the invention is concerned with the use of such combinations in the treatment of cancer. The present invention also relates to pharmaceutical compositions containing such combinations.

5,6-Dimethylxanthenone-4-acetic acid (DMXAA) is represented by the following formula:

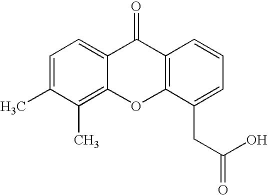

Three phase I clinical trials of DMXAA as a monotherapy have recently been completed, with dynamic MRI showing that it induces a significant reduction in tumour blood flow at well-tolerated doses. DMXAA is thus one of the first vascular disrupting agents (VDAs) for which activity (irreversible inhibition of tumour blood flow) has been documented in human tumours. These findings are in agreement with preclinical studies using syngeneic murine tumours or human tumour xenografts which showed that its antivascular activity produced prolonged inhibition of tumour blood flow leading to extensive regions of haemorrhagic necrosis.

However, in these phase I clinical trials of DMXAA there were very few tumour responses, demonstrating that DMXAA alone does not have significant potential in cancer treatment as a single agent. Therefore, there is a need to identify compounds that could have a synergistic effect with DMXAA.

There is a new class of cancer drugs available that are not cytotoxics, but block the growth factor signalling pathways. Examples include Avastin™ (bevacizumab), a humanised monoclonal antibody that binds to vascular endothelial growth factor (VEGF). By doing so, it inhibits angiogenesis (growth of new blood vessels), starving growing tumour of nutrients. We have surprisingly found that DMXAA may act synergistically with these new agents, enhancing their anti-cancer activity.

### Vascular Endothelial Growth Factor

Tumours have been found to overexpress certain growth factors that enable them to proliferate rapidly. Chief among these is VEGF. Tumours secrete VEGF, which stimulates endothelial proliferation and migration through two high-affinity receptor-associated tyrosine kinases found primarily on the vascular endothelium, VEGF-R1 (Flt-1) and VEGF-R2 (Flk-1/KDR). Expression levels of VEGF are negatively correlated with prognosis and survival in cancer, and inhibiting its binding to its receptor has been shown to improve survival.

VEGF is targeted by Avastin™ (bevacizumab, a humanised monoclonal antibody marketed by Genentech in the US and Roche elsewhere). The antibody binds directly to VEGF, preventing it from binding to VEGF receptors on the vascular endothelium. This means that the new blood vessels required by the tumour do not develop, and it cannot grow. Avastin™ combined with standard chemotherapy has been shown to offer a survival advantage over standard chemotherapy alone in colorectal, lung and breast cancers in phase III trials.

### Previous DMXAA Combination Studies

DMXAA has previously been demonstrated to have synergy with a number of agents in xenograft studies. These agents include widely used cytotoxic chemotherapies such as taxanes (paclitaxel and docetaxel), platins (cisplatin and to carboplatin), vinca alkaloids (vincristine), antimetabolites (gemcitabine), topoisomerase II inhibitors (etoposide) and anthracyclines (doxorubicin). It is believed that the synergy arises because DMXAA causes necrosis in the centre of tumours by disrupting the blood vessels that supply the core, but it leaves a viable rim of rapidly proliferating cancer cells that are supplied by normal blood vessels. These remaining malignant cells are targeted by the cytotoxic agents, which primarily act by disrupting cell division in various ways.

DMXAA is currently in two phase II trials examining its anti-tumour efficacy in combination with paclitaxel and carboplatin, and one trial combining it with docetaxel. Although the taxanes are believed to have anti-angiogenic properties, this is via a very different mechanism from the growth factor inhibitors. The cytotoxic effect of the taxanes is caused by interference with tubulin, which prevents normal mitosis (cell division). This is the main effect seen at the high doses of the taxanes used in cancer chemotherapy. A secondary effect is disruption of newly formed blood vessels, since the cells of the new vascular endothelium depend on tubulin to maintain their shape. However, this effect is normally seen only at doses too low to be cytotoxic. Any synergy between DMXAA and the taxanes is thought to be a result of the targeting of different parts of the tumour, as described above, rather than due to its anti-angiogenic properties.

Other agents have also been shown to enhance the activity of DMXAA in xenograft studies. Although the exact mechanism of action of DMXAA is not understood, it is believed to cause upregulation of various cytokines, and compounds with similar activity appear to enhance its effectiveness. These include tumour necrosis factor stimulating compounds and immunomodulatory compounds such as intracellular adhesion molecules (ICAMs).

Diclofenac, an NSAID that has been shown to enhance the anti-tumour activity of DMXAA, is believed to affect the PK of DMXAA via competition for metabolic pathways. At a concentration of 100 μM, diclofenac has been shown to significantly inhibit glucoronidation (>70%) and 6-methylhydroxylation (>54%) to of DMXAA in mouse and human liver microsomes. In vivo, diclofenac (100 mg/kg i.p.) has been shown to result in a 24% and 31% increase in the plasma DMXAA AUC (area under the plasma concentration-time curve) and a threefold increase in T_{1/2} (P<0.05) in male and female mice respectively (Zhou et al. (2001) Cancer Chemother. Pharmacol. 47, 319-326). Other NSAIDs have been shown to have a similar effect.

Similarly to diclofenac, thalidomide, which is approved for erythema nodosum leprosum (ENL), seems to enhance the activity of DMXAA. It competes for glucuronidation, prolonging DMXAA's presence at therapeutic levels in tumour tissue. Thalidomide increases the AUC of DMXAA by 1.8 times in plasma, liver and spleen and by three times in tumour (Kestell et al. (2000) Cancer Chemother. Pharmacol. 46(2), 135-41). Thalidomide is known to have anti-angiogenic effects, but these are not believed to be responsible for its synergy with DMXAA. It would not be expected that combining with vascular endothelial growth factor binder would have a similar effect to that of thalidomide on the effectiveness of DMXAA.

### Previous Vascular Endothelial Growth Factor Binder Combination Studies

Clinical evidence teaches away from combining different types of vascular targeting agents. It has been shown that Avastin™ does not have a synergistic effect when used in combination with thalidomide, an angiogenesis inhibitor, in metastatic renal cell carcinoma (Elaraj et al. (2004) J. Immunother. 27(4) (Jul.-Aug.), 259-64). Progression-free survival was the same in patients treated with Avastin™ alone or Avastin™ combined with thalidomide.

In its approved indication, colorectal cancer, Avastin™ is used in combination with 5-FU (5-fluorouracil), which does not have anti-angiogenic properties. Avastin™ has also been shown to improve median survival in breast and lung cancer patients when combined with paclitaxel. Although paclitaxel does have some anti-angiogenic properties, its primary mechanism of action in the high doses in which it is used for cancer treatment is as a cytotoxic, as described above. Therefore, this would not suggest that DMXAA would have a similar synergy with Avastin™, since DMXAA is very unlike paclitaxel in its mechanism of action and is not a cytotoxic.

### DESCRIPTION OF THE INVENTION

In a first aspect, the present invention provides a method for modulating neoplastic growth, which comprises administering to a mammal, including a human, in need of treatment a compound of formula (I):

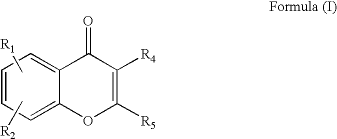

wherein:
(a) R₄ and R₅ together with the carbon atoms to which they are joined, form a 6-membered aromatic ring having a substituent —R₃ and a radical —(B)—COOH where B is a linear or branched substituted or unsubstituted C₁-C₆ alkylene radical, which is saturated or ethylenically unsaturated, and wherein R₁, R₂ and R₃ are each independently selected from the group consisting of H, C₁-C₆ alkyl, halogen, CF₃, CN, NO₂, NH₂, OH, OR^{a}, NHCOR^{b}, NHSO₂R^{c}, SR^{d}, SO₂R^{e} or NHR^{f}, wherein each of R^{a}, R^{b}, R^{c}, R^{d}, R^{e} and R^{f} is independently C₁-C₆ alkyl optionally substituted with one or more substituents selected from hydroxy, amino and methoxy; or
(b) one of R₄ and R₅ is H or a phenyl radical, and the other of R₄ and R₅ is H or a phenyl radical which may optionally be substituted, thienyl, furyl, naphthyl, a C₁-C₆ alkyl, cycloalkyl, or aralkyl radical; R₁ is H or a C₁-C₆ alkyl or C₁-C₆ alkoxy radical; R₂ is the radical —(B)—COOH where B is a linear or branched substituted or unsubstituted C₁-C₆ alkylene radical, which is saturated or ethylenically unsaturated,
   or a pharmaceutically acceptable salt, ester or prodrug thereof and concomitantly or sequentially administering a vascular endothelial growth factor binder.

Where (B) in the radical —(B)—COOH is a substituted C₁-C₆ alkyl radical, the substituents may be alkyl, for example methyl, ethyl, propyl or isopropyl, or halide such as fluoro, chloro or bromo groups. A particularly preferred substituent is methyl.

In one embodiment of the first aspect of the invention, the compound of the formula (I) as defined above is a compound of the formula (II):

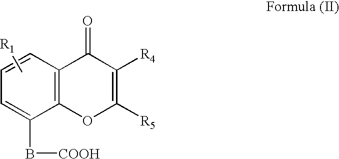

where R₁, R₄, R₅ and B are as defined above for formula (I) in part (b).

In a further embodiment of the first aspect of the invention, the compound of formula (I) as defined above is a compound of the formula (III):

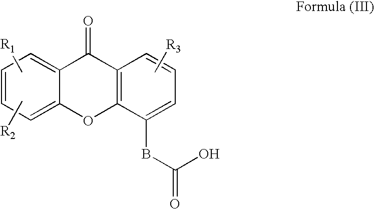

wherein R₁, R₂ and R₃ are each independently selected from the group consisting of H, C₁-C₆ alkyl, halogen, CF₃, CN, NO₂, NH₂, OH, OR^{a}, NHCOR^{b}, NHSO₂R^{c}, SR^{d}, SO₂R^{e} or NHR^{f}, wherein each of R^{a}, R^{b}, R^{c}, R^{d}, R^{e} and R^{f} is independently C₁-C₆ alkyl optionally substituted with one or more substituents selected from hydroxy, amino and methoxy;
wherein B is as defined for formula (I) above;
and wherein in each of the carbocyclic aromatic rings in formula (I), up to two of the methine (—CH═) groups may be replaced by an aza (—N═) group;
and wherein any two of R₁, R₂ and R₃ may additionally together represent the group —CH═CH—CH═CH—, such that this group, together with the carbon or nitrogen atoms to which it is attached, forms a fused 6 membered aromatic ring.

For example, the compound of formula (III) may be a compound of the formula (IV):

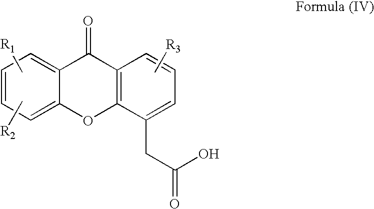

wherein R, R₁, R₂ and R₃ are as defined for formula (III).

In one embodiment of the compound of formula (IV), R₂ is H, one of R₁ and R₃ is selected from the group consisting of C₁-C₆ alkyl, halogen, CF₃, CN, NO₂, NH₂, OH, OR^{a}, NHCOR^{b}, NHSO₂R^{c}, SR^{d}, SO₂R^{e} or NHR^{f}, wherein each of R^{a}, R^{b}, R^{c}, R^{d}, R^{e} and R^{f} is independently C₁-C₆ alkyl optionally substituted with one or more substituents selected from hydroxy, amino and methoxy, and the other of R₁ and R₃ is H.

In one embodiment, in the compound of formula (I) R₄ is H or a phenyl radical, R₅ is H or a phenyl radical which may optionally be substituted, thienyl, furyl, naphthyl, a C₁-C₆ alkyl, cycloalkyl, or aralkyl radical; R₁ is H or a C₁-C₆ alkyl or C₁-C₆ alkoxy radical; R₂ is radical —(B)—COOH where B is a linear or branched substituted or unsubstituted C₁-C₆ alkylene radical, which is saturated or ethylenically unsaturated.

For example, the compound of formula (IV) may be a compound of the formula (V):

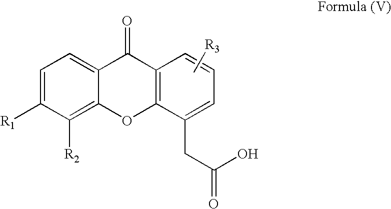

wherein R, R₁, R₂ and R₃ are as defined for formula (IV).

The compound of formula (V) may be, for example, 5,6-dimethylxanthenone-4-acetic acid (DMXAA).

Pharmaceutically-acceptable salts include acid addition salts and base addition salts. Such salts may be formed by conventional means, for example by reaction of a free acid or a free base form of a compound of formula (I) with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

In another aspect, the present invention provides the use of a vascular endothelial growth factor binder for the manufacture of a medicament (e.g. a unit dose of the medicament), for simultaneous, separate or sequential administration with a compound of formula (I) as defined above or a pharmaceutically acceptable salt, ester or prodrug thereof (e.g. a unit dose of the compound of formula (I) as defined above or a pharmaceutically acceptable salt, ester or prodrug thereof), for the modulation of neoplastic growth.

In a further aspect, the invention provides the use of a compound of formula (I) as defined above or a pharmaceutically acceptable salt or ester thereof for the manufacture of a medicament (e.g. a unit dose of the medicament), for simultaneous, separate or sequential administration with a vascular endothelial growth factor binder (e.g. a unit dose of the vascular endothelial growth factor binder), for the modulation of neoplastic growth.

According to one aspect, the neoplastic growth is a tumour and/or a cancer.

In a further aspect, the cancer is one or more of ovarian, prostate, lung, colorectal, breast, pancreatic and renal cancer.

In a further aspect, there is provided a pharmaceutical formulation (e.g. in a unit dose) comprising a combination of a compound of formula (I) as defined above or a pharmaceutically acceptable salt or ester or prodrug thereof (e.g. in a unit dose) and a vascular endothelial growth factor binder (e.g. in a unit dose).

In one embodiment there is provided a compound according to formula (I) or a pharmaceutically acceptable salt, ester or prodrug thereof and a vascular endothelial growth factor binder for use (in combination) as a medicament for the modification of neoplastic growth.

Furthermore, the invention also provides a kit comprising in combination for simultaneous, separate or sequential use in modulating neoplastic growth, a compound of formula (I) as defined above or a pharmaceutically acceptable salt or ester or prodrug thereof and a vascular endothelial growth factor binder.

The compound of formula (I) as defined above or pharmaceutically acceptable salt or ester or prodrug thereof and the vascular endothelial growth factor binder may be administered sequentially or concomitantly. For example, the compound of formula (I) as defined above or pharmaceutically acceptable salt, ester or prodrug thereof and the vascular endothelial growth factor binder may be administered concomitantly.

In one embodiment, the pharmaceutically acceptable salt is a sodium salt.

The compound of formula (I) as defined above or pharmaceutically acceptable salt, ester or prodrug thereof and the vascular endothelial growth factor binder may be administered simultaneously, separately or sequentially.

In one embodiment, the vascular endothelial growth factor binder is a monoclonal antibody.

In a further embodiment, vascular endothelial growth factor binder (VEGF) is Avastin™ (bevacizumab).

The amount of a combination of a compound of formula (I) as defined above or pharmaceutically acceptable salt, ester or prodrug thereof and a vascular endothelial growth factor binder required to be effective as a modulator of neoplastic growth, or a combination that further comprises a taxane, will, of course vary and is ultimately at the discretion of the medical practitioner. The factors to be considered include the route of administration and nature of the formulation, the mammal's bodyweight, age and general condition and the nature and severity of the disease to be treated.

A suitable effective dose of a compound of formula (I) as defined above, or a pharmaceutically acceptable salt, ester or prodrug thereof, for administration, concomitantly or sequentially, with a vascular endothelial growth factor binder, for the treatment of cancer is in the range of 600 to 4900 mg/m². For example from 2500 to 4000 mg/m², for example from 1200 to 3500 mg/m², for example from 2000 to 3000 mg/m², for example from 1200 to 2500 mg/m², for example from 2500 to 3500 mg/m², for example from 2250 to 2750 mg/m².

A suitable effective dose of vascular endothelial growth factor binder, for administration concomitantly or sequentially with a compound of formula (I) as defined above or pharmaceutically acceptable salt, ester or prodrug thereof for the treatment of cancer is in the range of 1-10 mg/kg, for example about 5 mg/kg.

In a further embodiment, a suitable effective dose of vascular endothelial growth factor binder, for administration concomitantly or sequentially with a compound of formula (I) as defined above or pharmaceutically acceptable salt, ester or prodrug thereof for the treatment of cancer is in the range from 1 to 30 mg/kg, for example from about 10 to about 20 mg/kg and more particularly about 15 mg/kg.

A compound of formula (I) as defined above or pharmaceutically acceptable salt, ester or prodrug thereof and the vascular endoethelial growth factor binder may be administered in any suitable form, for example in the form of a pharmaceutical formulation.

Pharmaceutical formulations comprise the active ingredients (that is, the combination of a compound of formula (I) as defined above or pharmaceutically acceptable salt, ester or prodrug thereof and the vascular endothelial growth factor binder, for example together with one or more pharmaceutically acceptable carriers therefor and optionally other therapeutic and/or prophylactic ingredients. The carrier(s) must be acceptable in the sense of being compatible with the other ingredients in the formulation and not deleterious to the recipient thereof.

Accordingly, the present invention provides a pharmaceutical formulation comprising a combination of a compound of formula (I) as defined above or pharmaceutically acceptable salt, ester or prodrug thereof (e.g. a unit dose of a compound of formula (I) as defined above or pharmaceutically acceptable salt, ester or prodrug thereof) and a vascular endothelial growth factor binder (e.g. a unit dose of the vascular endothelial growth factor binder), for example in association with one or more pharmaceutically acceptable carriers therefor.

The invention further provides a process for the preparation of a pharmaceutical formulation which process comprises bringing into association a combination of a compound of formula (I) as defined above or a pharmaceutically acceptable salt, ester or prodrug thereof (e.g. a unit dose of a compound of formula (I) as defined above or pharmaceutically acceptable salt, ester or prodrug thereof) and a vascular endothelial growth factor binder (e.g. a unit dose of the vascular endothelial growth factor binder) optionally together with one or more pharmaceutically acceptable carriers therefor in. For example, the pharmaceutical formulation may be in a unit dose.

The pharmaceutical formulation may be delivered intravenously. The pharmaceutical formulation for intravenous administration may be used in the form of sterile aqueous solutions or in an oleaginous vehicle which may contain other substances, for example, enough salts or glucose to make the solution isotonic with blood. The aqueous solutions may be buffered (e.g. to a pH from 3 to 9), if necessary.

As used herein, the term “prodrug” includes entities that have certain protected group(s) and which may not possess pharmacological activity as such, but may, in certain instances, be administered (such as orally or parenterally) and thereafter metabolised in the body to form the agent which are pharmacologically active.

Further anti-cancer agents or therapies may be used in conjunction with the combination of a compound of formula (I) (e.g. DMXAA) and a vascular endothelial growth factor binder (e.g. bevacizumab). Particular anti-cancer agents that may be mentioned in this respect include taxanes. Thus, further embodiments of the invention include the following (in which embodiments, references to compounds of formula (I) include references to compounds of formula (II), (III), (IV) or (V)).
(A) A method for modulating neoplastic growth, which method comprises administering to a mammal, including a human, in need of such treatment a compound of formula (I), as hereinbefore defined, or a pharmaceutically acceptable salt, ester or prodrug thereof and concomitantly or sequentially administering:
   (i) a vascular endothelial growth factor binder; and
   (ii) a taxane.
(B) The use of a vascular endothelial growth factor binder for the manufacture of a medicament (e.g. a unit dose of the medicament) for simultaneous, separate or sequential administration with:
   (i) a compound of formula (I), as hereinbefore defined, or a pharmaceutically acceptable salt, ester or prodrug thereof (e.g. a unit dose of the compound of formula (I), as hereinbefore defined, or a pharmaceutically acceptable salt, ester or prodrug thereof); and
   (ii) a taxane (e.g. a unit dose of the taxane),
   for the modulation of neoplastic growth.
(C) The use of a compound of formula (I), as hereinbefore defined, or a pharmaceutically acceptable salt, ester or prodrug thereof for the manufacture of a medicament (e.g. a unit dose of the medicament) for simultaneous, separate or sequential administration with:
   (i) a vascular endothelial growth factor binder (e.g. a unit dose of the vascular endothelial growth factor binder); and
   (ii) a taxane (e.g. a unit dose of the taxane),
   for the modulation of neoplastic growth.
(D) The use of a taxane for the manufacture of a medicament (e.g. a unit dose of the medicament) for simultaneous, separate or sequential administration with:
   (i) a vascular endothelial growth factor binder (e.g. a unit dose of the vascular endothelial growth factor binder); and
   (ii) a compound of formula (I), as hereinbefore defined, or a pharmaceutically acceptable salt, ester or prodrug thereof (e.g. a unit dose of the compound of formula (I), as hereinbefore defined, or a pharmaceutically acceptable salt, ester or prodrug thereof),
   for the modulation of neoplastic growth.
(E) A pharmaceutical formulation (e.g. in a unit dose) comprising a combination of a compound of formula (I), as hereinbefore defined, or a pharmaceutically acceptable salt, ester or prodrug thereof (e.g. in a unit dose), a vascular endothelial growth factor binder (e.g. in a unit dose) and a taxane (e.g. in a unit dose).
(F) A compound of formula (I), as hereinbefore defined, or a pharmaceutically acceptable salt, ester or prodrug thereof, a vascular endothelial growth factor binder and a taxane for use (in combination) as a medicament for the modification of neoplastic growth.
(G) A kit comprising in combination for simultaneous, separate or sequential use in modulating neoplastic growth:
   (i) a compound of formula (I), as hereinbefore defined, or a pharmaceutically acceptable salt or ester or prodrug thereof;
   (ii) a vascular endothelial growth factor binder; and
   (iii) a taxane.
(H) A process for the preparation of a pharmaceutical formulation as defined at (E) above, which process comprises bringing into association a combination of a compound of formula (I), as hereinbefore defined, or a pharmaceutically acceptable salt, ester or prodrug thereof (e.g. a unit dose of a compound of formula (I) as defined above or pharmaceutically acceptable salt, ester or prodrug thereof), a vascular endothelial growth factor binder (e.g. a unit dose of the vascular endothelial growth factor binder) and a taxane (e.g. a unit dose of the taxane), optionally together with one or more pharmaceutically acceptable carriers therefor.

In the above embodiments of the invention, the taxane may, in particular, be paclitaxel or docetaxel.

In relation to the above embodiments of the invention, a suitable effective dose of taxane (e.g. paclitaxel), for administration concomitantly or sequentially with a compound of formula (I) as defined above or pharmaceutically acceptable salt, ester or prodrug thereof and a vascular endothelial growth factor binder for the treatment of cancer is in the range from 1 to 10 mg/kg, for example from about 4 to about 5 mg/kg.

Alternatively, a suitable effective dose of taxane (e.g. paclitaxel) is in the range of 100 to 250 mg/m², such as from about 175 to about 200 mg/m².

### DESCRIPTION OF THE FIGURES

FIG. 1: shows the average tumour volume (relative to the average volume on the first day of treatment) for HT29 (colorectal) xenografts observed for an untreated control group of mice and for mice given (i.e. treated with) Avastin™ (alone), DMXAA (alone), or a combination of Avastin™ and DMXAA.

FIG. 2: is a representation of the same data used to generate FIG. 1, but expressed in terms of the percentage of mice having tumour volume less than four times the volume measured on the first day of treatment.

FIGS. 3 and 4: show equivalent data to FIGS. 1 and 2, respectively, but for A549 (lung carcinoma) xenografts.

FIG. 5: shows the average tumour volume (relative to the average volume on the first day of treatment) for A549 (lung carcinoma) xenografts observed for an untreated control group of mice and for mice given (i.e. treated with) Avastin™ (alone), DMXAA (alone), paclitaxel (alone) or a combination of Avastin™ paclitaxel and DMXAA.

FIG. 6: is a representation of the same data used to generate FIG. 5, but expressed in terms of the percentage of mice having tumour volume less than four times the volume measured on the first day of treatment.

### EXAMPLES

### Example 1

### Method

Xenografts for human lung and colorectal cancers are set-up in groups of nude, athymic mice. The cell lines selected are HT29 (ATCC number HTB-38), a colorectal adenocarcinoma, and A549 (ATCC number CCL-185), a lung carcinoma.

The A549 and HT29 cell lines are selected as DMXAA has previously been shown to be effective in these cell lines when used in combination with paclitaxel or 5-FU in xenograft studies. In addition, Avastin™ is currently approved for treatment of colorectal cancer in combination with 5-FU and approval is being sought for use on breast and non-small cell lung carcinoma.

| | Cell | | Dose level | No. of |
|---|---|---|---|---|
| Group | line | Treatment | (mg/kg) | mice |
| 1 | A549 | Untreated control | — | 10 |
| 2 | A549 | DMXAA | 21 | 10 |
| 3 | A549 | Avastin ™ | 5 | 10 |
| 4 | A549 | DMXAA + | 21 & 5 | 10 |
| | | Avastin ™ | | |
| 5 | HT29 | Untreated control | — | 10 |
| 6 | HT29 | DMXAA | 21 | 10 |
| 7 | HT29 | Avastin ™ | 5 | 10 |
| 8 | HT29 | DMXAA + | 21 & 5 | 10 |
| | | Avastin ™ | | |

DMXAA has been given previously using a day (D) 0, 4 and 8 schedule when used in combination with paclitaxel or docetaxel. For this study, DMXAA is given twice in each of Weeks 1 and 4 of the study. Avastin™ is given twice weekly for four weeks.

Xenografts are measured two or three times per week and their absolute volume recorded; xenograft tumour volume relative to that recorded on Day 0 (V₀) is then calculated. The time taken to reach a relative tumour volume of 3× V₀ is used as a surrogate marker for survival.

### Results

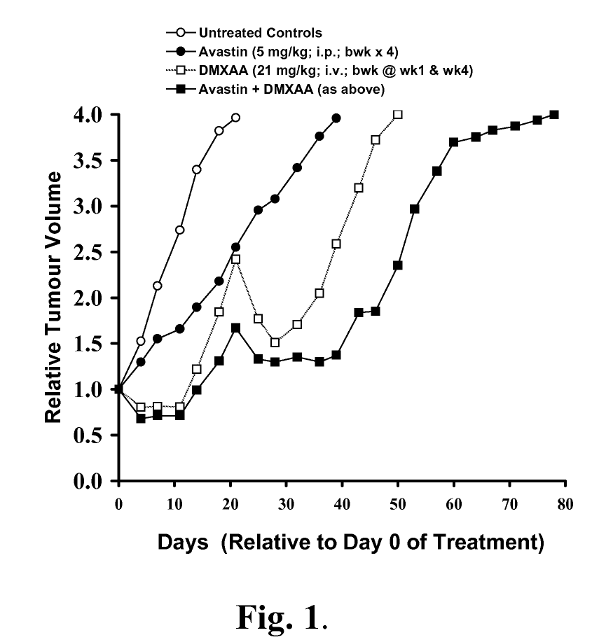

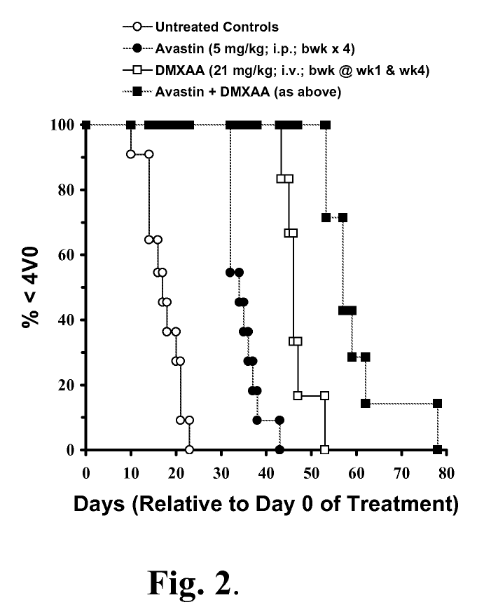

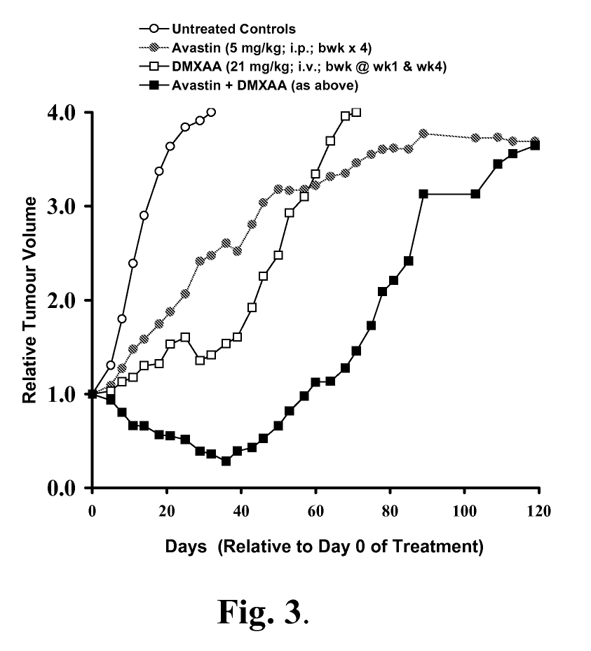

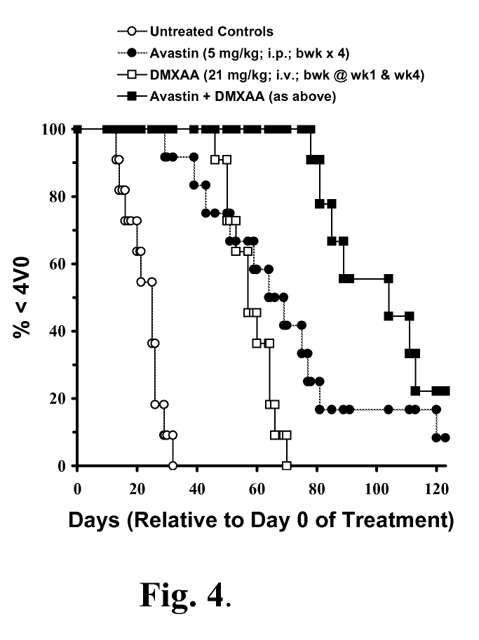

Tables 1A, 1B, 2A and 2B below, as well as FIGS. 1 to 4 show that the combination of Avastin™ and DMXAA provides an unexpected synergistic effect in delaying tumour growth.

TABLE 1A
Results of studies with HT29 xenografts.

| | | | | Tumour | | |
|---|---|---|---|---|---|---|
| | | | | Growth | Regression | |
| | Dose (mg/kg by | Drug | Median VQT | Delay^{a1} | Duration^{b1} | TTP^{c1} |
| Group | injection) | deaths | (Days) | (Days) | (Days) | (Days) |
| Untreated | — | — | 17 | — | 0 | 4 |
| Controls | | | | | | |
| Avastin ™ | 5 | 0/11 | 34 | 17 | 0 | 4 |
| DMXAA | 21 | 5/11 | 46 | 29 | 10 | 16 |
| Avastin ™/ | 5 + 21 | 4/11 | 57 | 40 | 10 | 18 |
| DMXAA | | | | | | |

^{a1}The difference in days for treated versus control tumours to quadruple in volume (control tumours quadrupled in 17 days).
^{b1}Tumour regression duration is the number of days that the tumour volume is less than the original treatment volume.
^{c1}TTP: Median time to disease progression

TABLE 1B
Results of studies with HT29 xenografts.

| | |
|---|---|
| Dose (mg/kg | Response^{d1} |

| Group | by injection) | PD | PR | SD | CR |
|---|---|---|---|---|---|
| Untreated | — | 0 | 0 | 0 | 0 |
| Controls | | | | | |
| Avastin ™ | 5 | 11 | 0 | 0 | 0 |
| DMXAA | 21 | 5 | 1 | 0 | 0 |
| Avastin ™/ | 5 + 21 | 6 | 1 | 0 | 0 |
| DMXAA | | | | | |

^{d1}PD: Progressive Disease (≧50% increase in tumour size) PR: Partial Response (≧50% reduction in tumour size sustained over two weeks) SD: Stable Disease (does not satisfy criteria for PR of PD) CR: Complete Response (cure; undetectable tumour over two weeks)

TABLE 2A
Results of studies with A549 xenografts.

| | | | | Tumour | | |
|---|---|---|---|---|---|---|
| | | | | Growth | Regression | |
| | Dose (mg/kg by | Drug | Median VQT | Delay^{a2} | Duration^{b2} | TTP^{c2} |
| Group | injection) | deaths | (Days) | (Days) | (Days) | (Days) |
| Untreated | — | — | 25 | — | 0 | 5 |
| Controls | | | | | | |
| Avastin ™ | 5 | 0/12 | 67 | 42 | 0 | 8 |
| DMXAA | 21 | 1/12 | 57 | 32 | 0 | 14 |
| Avastin ™/ | 5 + 21 | 2/12 | 104 | 79 | 52 | 68 |
| DMXAA | | | | | | |

^{a2}The difference in days for treated versus control tumours to quadruple in volume (control tumours quadrupled in 25 days).
^{b2}Tumour regression duration is the number of days that the tumour volume is less than the original treatment volume.
^{c2}TTP: Median time to disease progression

TABLE 2B
Results of studies with A459 xenografts.

| | |
|---|---|
| Dose (mg/kg | Response^{d2} |

| Group | by injection) | PD | PR | SD | CR |
|---|---|---|---|---|---|
| Untreated | — | 0 | 0 | 0 | 0 |
| Controls | | | | | |
| Avastin ™ | 5 | 11 | 1 | 0 | 0 |
| DMXAA | 21 | 11 | 0 | 0 | 0 |
| Avastin ™/ | 5 + 21 | 2 | 7 | 1 | 0 |
| DMXAA | | | | | |

^{d2}PD: Progressive Disease (≧50% increase in tumour size) PR: Partial Response (≧50% reduction in tumour size sustained over two weeks) SD: Stable Disease (does not satisfy criteria for PR of PD) CR: Complete Response (cure; undetectable tumour over two weeks)

### Example 2

### Method

The experimental set-up of this example with respect to the xenografts, mice and cell line is as described in Example 1 above.

| | Cell | | Dose level | No. of |
|---|---|---|---|---|
| Group | line | Treatment | (mg/kg) | mice |
| 1 | A549 | Untreated control | — | 11 |
| 2 | A549 | DMXAA | 21 | 11 |
| 3 | A549 | Avastin ™ | 5 | 11 |
| 4 | A549 | Paclitaxel | 5 | 11 |
| 5 | A549 | DMXAA + | 21, 5 & 5 | 11 |
| | | Paclitaxel + | | |
| | | Avastin ™ | | |

DMXAA has been given previously using a day (D) 0, 4 and 8 schedule when used in combination with paclitaxel or docetaxel. For this study, DMXAA is given twice in each of Weeks 1 and 4 of the study. Avastin™ is given twice weekly for four weeks. For this study, Paclitaxel is given twice in each of Weeks 1 and 4 of the study.

Xenografts are measured two or three times per week and their absolute volume recorded; xenograft tumour volume relative to that recorded on Day 0 (V₀) is then calculated. The time taken to reach a relative tumour volume of 3× V₀ is used as a surrogate marker for survival.

### Results

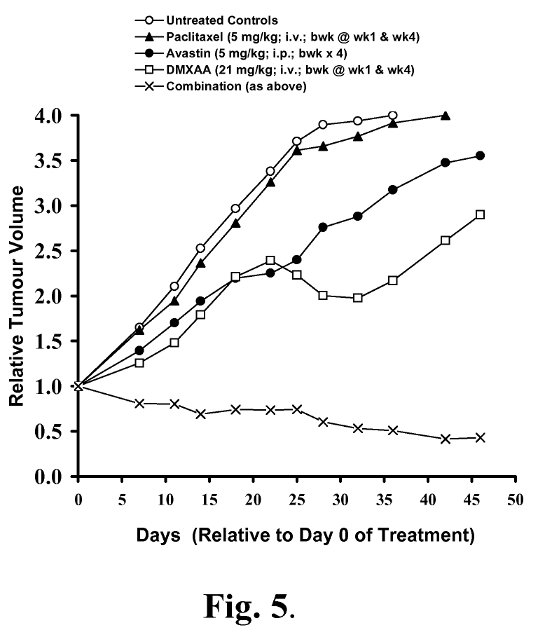

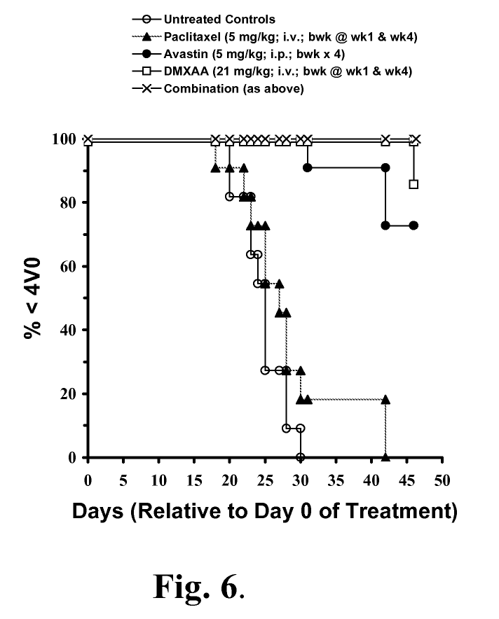

Tables 3A and 3B below, as well as FIGS. 5 and 6 show that the combination of Avastin™, Paclitaxel and DMXAA provides an unexpected synergistic effect in delaying tumour growth.

TABLE 3A
Results of studies with A549 xenografts.

| | | | | Tumour | | |
|---|---|---|---|---|---|---|
| | | | | Growth | Regression | |
| | Dose (mg/kg by | Drug | Median VQT | Delay^{a3} | Duration^{b3} | TTP^{c3} |
| Group | injection) | deaths | (Days) | (Days) | (Days) | (Days) |
| Untreated | — | — | 25 | — | 0 | 7 |
| Controls | | | | | | |
| Paclitaxel | 5 | 0/11 | 28 | 3 | 0 | 7 |
| Avastin ™ | 5 | 0/11 | >42 | >17 | 0 | 7 |
| DMXAA | 21 | 4/11 | >46 | >21 | 0 | 7 |
| Paclitaxel/ | 5 + 5 + 21 | 1/11 | >46 | >46 | >46 | 42 |
| Avastin ™/ | | | | | | |
| DMXAA | | | | | | |

^{a3}The difference in days for treated versus control tumours to quadruple in volume (control tumours quadrupled in 25 days).
^{b3}Tumour regression duration is the number of days that the tumour volume is less than the original treatment volume.
^{c3}TTP: Median time to disease progression.

TABLE 3B
Results of studies with A549 xenografts.

| | |
|---|---|
| Dose (mg/kg | Response^{d3} |

| Group | by injection) | PD | PR | SD | CR |
|---|---|---|---|---|---|
| Untreated | — | 11 | 0 | 0 | 0 |
| Controls | | | | | |
| Paclitaxel | 5 | 11 | 0 | 0 | 0 |
| Avastin ™ | 5 | 11 | 0 | 0 | 0 |
| DMXAA | 21 | 7 | 0 | 0 | 0 |
| Paclitaxel/ | 5 + 5 + 21 | 0 | 4 | 4 | 2 |
| Avastin ™/ | | | | | |
| DMXAA | | | | | |

^{d3}PD: Progressive Disease (≧50% increase in tumour size) PR: Partial Response (≧50% reduction in tumour size sustained over two weeks) SD: Stable Disease (does not satisfy criteria for PR of PD) CR: Complete Response (cure; undetectable tumour over two weeks)

### Abbreviations

AUC=area under plasma concentration curve
CR=Complete Response
DMXAA=5,6-dimethylxanthenone-4-acetic acid
ENL=erythema nodosum leprosum
5-FU=5-fluorouracil
ICAM=intracellular adhesion molecule
i.p.=intraperitoneal
MRI=magnetic resonance imaging
NSAID=non-steroidal anti-inflammatory drug
PD=Progressive Disease
PK=pharmacokinetics
PR=Partial Response
SD=Stable Disease
VEGF=vascular endothelial growth factor
VDA=vascular disrupting agent
VQT=(tumour) volume quadrupling time

1. A method for modulating neoplastic growth, which comprises administering to a mammal, including a human, in need of treatment a compound of Formula (I): 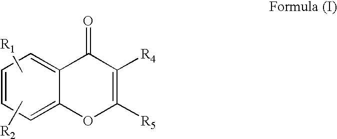
wherein:
(a) R₄ and R₅ together with the carbon atoms to which they are joined, form a 6-membered aromatic ring having a substituent —R₃ and a radical —(B)—COOH where B is a linear or branched substituted or unsubstituted C₁-C₆ alkylene radical, which is saturated or ethylenically unsaturated, and wherein R₁, R₂ and R₃ are each independently selected from the group consisting of H, C₁-C₆ alkyl, halogen, CF₃, CN, NO₂, NH₂, OH, OR^{a}, NHCOR^{b}, NHSO₂R^{c}, SR^{d}, SO₂R^{e} or NHR^{f}, wherein each of R^{a}, R^{b}, R^{c}, R^{d}, R^{e} and R^{f} is independently C₁-C₆ alkyl optionally substituted with one or more substituents selected from hydroxy, amino and methoxy; or
(b) one of R₄ and R₅ is H or a phenyl radical, and the other of R₄ and R₅ is H or a phenyl radical which may optionally be substituted, thienyl, furyl, naphthyl, a C₁-C₆ alkyl, cycloalkyl, or aralkyl radical; R₁ is H or a C₁-C₆ alkyl or C₁-C₆ alkoxy radical; R₂ is the radical —(B)—COOH where B is a linear or branched substituted or unsubstituted C₁-C₆ alkylene radical, which is saturated or ethylenically unsaturated,
or a pharmaceutically acceptable salt, ester or prodrug thereof and concomitantly or sequentially administering a vascular endothelial growth factor binder.

2. The method according to claim 1 wherein the compound of Formula (I) is a compound of Formula (II): 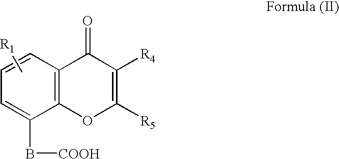
wherein R₁, R₄, R₅ and B are as defined for formula (I) in claim 1 part (b).

3. The method according to claim 1 wherein the compound of Formula (I) is a compound of Formula (III): 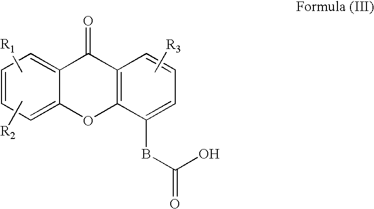
wherein R₁, R₂ and R₃ are each independently selected from the group consisting of H, C₁-C₆ alkyl, halogen, CF₃, CN, NO₂, NH₂, OH, OR^{a}, NHCOR^{b}, NHSO₂R^{c}, SR^{d}, SO₂R^{e} or NHR^{f}, wherein each of R^{a}, R^{b}, R^{c}, R^{d}, R^{e} and R^{f} is independently C₁-C₆ alkyl optionally substituted with one or more substituents selected from hydroxy, amino and methoxy;
wherein B is as defined for formula (I) in claim 1;
and wherein in each of the carbocyclic aromatic rings in formula (I), up to two of the methine (—CH═) groups may be replaced by an aza (—N═) group;
and wherein any two of R₁, R₂ and R₃ may additionally together represent the group —CH═CH—CH═CH—, such that this group, together with the carbon or nitrogen atoms to which it is attached, forms a fused 6 membered aromatic ring.

4. The method according to claim 3, wherein the compound of Formula (III) is a compound of Formula (IV): 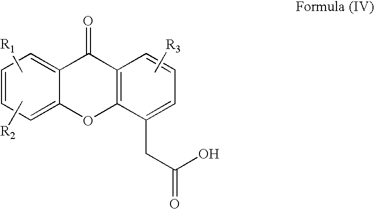
wherein R, R₁, R₂ and R₃ are as defined for formula (III) in claim 3.

5. The method according to claim 4 wherein the compound of Formula (IV) is a compound of Formula (V): 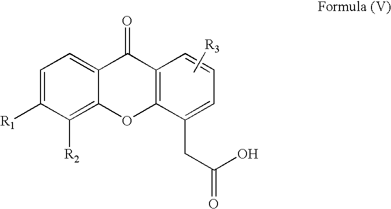
wherein R, R₁, R₂ and R₃ are as defined for formula (IV) in claim 4.

6. The method according to claim 1, wherein the compound of Formula (I) is DMXAA or a pharmaceutically acceptable salt, ester or prodrug thereof.

7. The method according to claim 1, which method further comprises administering to a mammal, including a human, in need of treatment a taxane.

8. A method according to claim 1 wherein the compound of formula (I) or a pharmaceutically acceptable salt, ester or prodrug thereof and the vascular endothelial growth factor binder are administered concomitantly.

9. A method according to claim 1 wherein the compound of formula (I) or pharmaceutically acceptable salt, ester or prodrug thereof and the vascular endothelial growth factor binder are administered sequentially.

10. The method according to claim 1 wherein the vascular endothelial growth factor binder is a monoclonal antibody.

11. The method according to claim 10 wherein the vascular endothelial growth factor binder is Avastin™ (bevacizumab).

12. The method according to any one of claims 7, 10 and 11 wherein the taxane is paclitaxel or docetaxel.

13. The method according to claim 1 wherein the method further comprises modulation of neoplastic growth in one of more of ovarian, prostate, lung, colorectal, pancreatic, breast and renal cancer.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. A pharmaceutical formulation comprising a combination of a compound of formula (I), (II), (III), (IV) or (V), as defined in any one of claims 1 to 6, or a pharmaceutically acceptable salt, ester or prodrug thereof, and a vascular endothelial growth factor binder.

26. The pharmaceutical formulation of claim 25 wherein the pharmaceutical formulation further comprises a pharmaceutically acceptable carrier.

27. A pharmaceutical formulation according to claim 25 wherein the formulation is adapted for intravenous administration.

28. A pharmaceutical formulation according to claim 25 wherein the vascular endothelial growth factor binder is bevacizumab.

29. A pharmaceutical formulation according to claim 25 wherein the compound of formula (I), (II), (III), (IV) or (V) is DMXAA or a pharmaceutically acceptable salt, ester or prodrug thereof.

30. A pharmaceutical formulation according to claim 25 further comprising a taxane.

31. A pharmaceutical formulation according to claim 30 wherein the taxane is paclitaxel or docetaxel.

32. A kit comprising, in combination for simultaneous, separate or sequential use in modulating neoplastic growth, a compound of formula (I), (II), (III), (IV) or (V), as defined in any one of claims 1 to 6, or a pharmaceutically acceptable salt, ester or prodrug thereof and a vascular endothelial growth factor binder.

33. The kit according to claim 32 wherein the growth factor inhibitor is bevacizumab.

34. The kit according to claim 32 wherein the compound of formula (I) is DMXAA or a pharmaceutically acceptable salt, ester or prodrug thereof.

35. The kit according to claim 32 further comprising, in combination for simultaneous, separate or sequential use in modulating neoplastic growth, a taxane.

36. The kit according to claim 35 wherein the taxane is paclitaxel or docetaxel.
